(19) United States
(12) Patent Application Publication
(10) Pub. No.: US 2008/0160628 A1
(43) Pub. Date: Jul. 3, 2008

(54) Method for the Determination of Degradable, Organic Carbon

(76) Inventors: Hans-Joachim Kupka, Neuberg (DE); Ralf Dunsbach, Neu-Anspach (DE)

Correspondence Address: MILDE & HOFFBERG, LLP, 10 BANK STREET, SUITE 460, WHITE PLAINS, NY, 10606, US

(21) Appl. No.: 11/885,660

(22) PCT Filed: Feb. 27, 2006
(86) PCT No.: PCT/EP2006/001764

§ 371 Date: Sep. 4, 2007

(30) Foreign Application Priority Data
Mar. 1, 2005 (DE) 10 2005 009 828.2

(51) Int. Cl. G01N 33/00

(52) U.S. Cl. 436/146

(57) ABSTRACT

A method for determining degradable, organic carbon comprises the following steps: the sample is fed to a combustion tube along with a carrier gas; the sample is treated with inert gas in the combustion tube at at least 850° C.; the obtained pyrolysis gas, which contains the AOC, is conducted across a metal oxide as an oxygen donor for oxidizing purposes without adding oxygen; the AOC is oxidized while the metal oxide is reduced; the AOC is measured by means of a detector while the CO₂ peak is detected, the carrier gas being switched from inert gas to O₂ once the CO₂ peak has passed; the RC is burned in the O₂ atmosphere while the metal oxide is regenerated; and the RC is measured by means of a detector.

The present invention relates to a method for the determination of degradable organic carbon.

On account of the waste disposal rules, the determination of the organic carbon content is, for many types of waste, decisive for their classification into corresponding landfill classes. When organic carbon is determined in the standard way according to DIN EN 13137, this will automatically reveal not only the organically bound carbon, but also the carbon present in elemental form. This elemental carbon is not subject to any biogenic change and does thus not impair the stability of the landfill. Therefore, an analytical method would be appreciated that makes a distinction between biodegradable and elemental carbon.

An approach to such an analytical method is the so-called VGB method. According to this method the sample to be analyzed is first subjected to pyrolysis, followed by high-temperature oxidation of the pyrolysis residue. The so-called residual carbon (RC) is then subtracted from the TOC content determined in the standard way, and the difference yields the so-called degradable or assimilable carbon (AOC). Since part of the degradable carbon is converted into elemental carbon during pyrolysis (pyrolysis coke), the AOC value is multiplied by an empirical factor. Normally, a value of 1.3 is here used. This method has two essential drawbacks in the laboratory process:
the classic TOC must be determined separately; this means the conduction of two analytical runs and thus twice as much work;
if RC>AOC, the AOC is prone to a significant statistic error due to the subtracting process as the absolute errors of the RC and TOC determination add up.

The direct determination of the RC and the AOC in an analytical process would therefore be appreciated.

It is the object of the present invention to provide, in consideration of the prior art and of the drawbacks shown above on the basis of the prior art, a method for determining degradable or assimilable organic carbon which permits a direct measurement of the AOC.

This object is achieved through a method for determining degradable organic carbon comprising the following method steps:
feeding the sample to a combustion tube together with a carrier gas;
treating the sample with inert gas in the combustion tube at at least 850° C.;
conducting the obtained pyrolysis gas, which contains the AOC, across a metal oxide as an oxygen donor for oxidizing purposes without adding oxygen;
oxidizing the AOC while the metal oxide is reduced; and
measuring the AOC by means of a detector while the CO₂ peak is detected;
switching the carrier gas from inert gas to O₂ once the CO₂ peak has passed;
burning the RC in the O₂ atmosphere while the metal oxide is regenerated; and
measuring the RC by means of a detector.

In this method the sample is first treated with inert gas at 850° C. The sample can here be fed to the combustion tube, preferably a vertical combustion tube, via a gripper which may simultaneously be designed and used as a metering lance. The resulting pyrolysis gas which contains the AOC (assimilable or degradable carbon) is passed across a tube which is heated to 930° C. and filled with CuO as the catalyst. The AOC is there oxidized, while CuO is reduced to CO₂, and is subsequently measured by means of a detector, preferably by means of an IR detector, and even more preferably by means of a non-dispersive IR detector. After the CO₂ peak has decayed, the carrier gas is switched from inert gas, preferably N₂, to O₂. In the O₂ atmosphere the RC (residual carbon) will then burn, while the metal oxide catalyst, preferably CuO, is regenerated. AOC and RC are directly determined with this method in one analytical run without the need for preparing the sample. This procedure thereby entails considerable advantages over the prior art.

Preferably, a vertically arranged combustion tube should be used, into which the sample is introduced. Such a vertically oriented combustion tube has the advantage for the method that the sample part can be completely purged with inert gas.

To achieve an automatic analytical procedure, the sample should be introduced by means of a gripper into the combustion tube. Furthermore, it is thereby ensured that each sample is introduced at the same speed into the tube, so that each sample passes through the same temperature gradient.

Preferably, the oxygen needed for combustion is directly supplied to the sample via a lance, on the end of which the gripper is arranged. The oxygen contained in the crucible is thereby removed prior to analysis.

Copper oxide should be used as the metal oxide; copper oxide is a very efficient oxygen donor over a wide temperature range.

If acidified samples are to be analyzed, the metal oxide used is preferably tungsten oxide.

Cerium dioxide should be used as the metal oxide whenever high chloride contents are present in the sample matrix.

Preferably, the AOC and/or the RC is/are measured by means of the IR detector because an IR detector is more selective than a coulometric detector.

The method according to the invention shall now be described with reference to the drawings, in which:

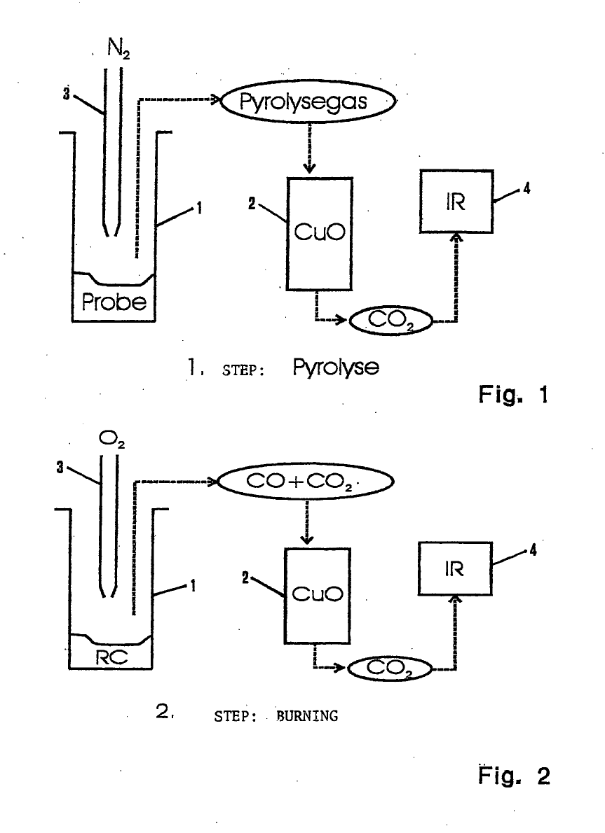

FIG. 1 shows a schematically illustrated equipment setup for performing the analytical step;

FIG. 2 shows the equipment setup schematically illustrated in FIG. 1 for performing the combustion step; and

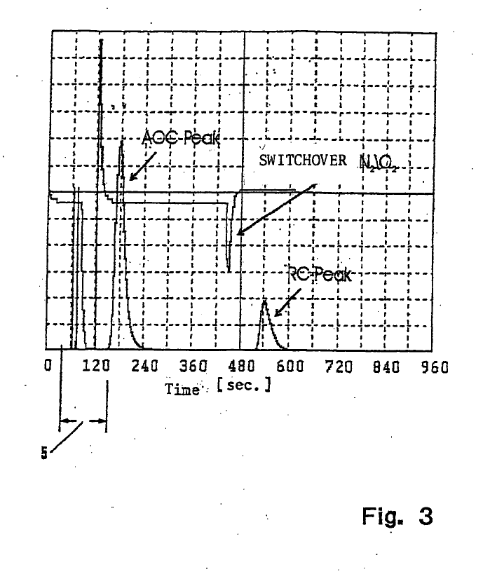

FIG. 3 is a time diagram in which the measurement peaks are shown.

The elemental analyzer “varioMAX CN” of the applicant, elementar Analysensysteme GmbH, 63452 Hanau, can be used as an equipment base for performing the method according to the invention, so that with respect to equipment details not listed individually in the following description, reference will be made to said analyzer.

The principal item of the apparatus is a combustion/pyrolysis assembly, as is schematically shown in FIG. 1 without combustion tubes.

The combustion unit (not shown) comprises two combustion tubes arranged one after the other. The first combustion tube is filled in the lower portion with CuO as the catalyst while the upper portion serves to receive the sample crucible that is shown in FIG. 1 and designated by reference numeral 1. The second combustion tube is filled with CuO as the oxidant, which is designated by reference numeral 2 in FIG. 1. The first tube (pyrolysis and combustion tube) is operated at 850° C. whereas the second tube (postoxidation) is operated at 930° C.

The sample which is filled into the crucible 1 is automatically introduced into the first combustion/pyrolysis tube by means of a gripper arm (not shown in more detail). The gas supply, illustrated by lance 3, is here carried out via the gripper which has integrated thereinto the illustrated lance 3, so that the sample is already exposed to the respective carrier gas (N₂) before being introduced into the furnace. The furnace temperature is adjustable, the upper limit being fixed to about 900° C. due to the use of CuO as catalyst material. If inert gas is present, the TOC is passed into the gas phase and oxidized on the CuO to obtain CO₂; the Cu is here reduced to Cu. The CO₂ is then measured in an IR detector 4 with a corresponding peak, which is shown in the diagram of FIG. 3.

After the first CO₂ peak has decayed, the inert gas is replaced by oxygen, fed again via the lance 3 directly to the residual carbon (RC), as outlined in FIG. 2. The residual carbon remaining in crucible 1 is thereby also converted to CO₂ and measured by analogy. Moreover, the CuO filling in the combustion tube is regenerated by the excess amount of O₂.

As can be seen in the diagram of FIG. 3, two clearly distinct peaks are thereby obtained for organically bound carbon (AOC peak) and inert carbon (RC peak). As can be inferred from the time scale (x-axis) of FIG. 3, the total measuring operation, including the sample supply (the area marked by reference numeral 5), takes about 10 minutes.

Incombustible constituents remain in the crucible 1, which drops into a reservoir after analysis. The crucible 1 itself can be reused after having been emptied.

This measurement sequence can be employed if the content of TIC (carbonates and hydrogen carbonates) is negligible in comparison with the TOC. If this is not the case, the sample could be acidified by means of HCl before. The sample must then be dried in the drying oven at 80° C. for 2 hours to expel the excess HCl. Subsequently, the sample can then be measured again, as has been described above. As an alternative, the TIC can be determined separately and deducted from the AOC value. Tests with CaCO₃ have shown that the TIC is completely converted to CO₂ during the pyrolysis phase.

### Measurement Results

With the above-described setup and the above-indicated procedure, different samples and pure substances have been measured. In the determination of the pure substances a correction factor must be used that, on the basis of comprehensive studies, was determined to be 1.3 for most organic compounds. This correction factor must be used for the AOC. The value for the AOC must be multiplied by this correction factor of 1.3. An exception to the above rule is humic acid obtained from the company Fluka, in the case of which the correction factor is calculated as a value of 2.6, on the assumption that said humic acid does not contain any elemental carbon.

The following tables list the measurement results of different substances subjected to comprehensive tests:

| | AOC | RC | AOC theor. | |
|---|---|---|---|---|
| Substance | [weight %] | [weight %] | [weight %] | Factor |
| Asparaginic | 26.0 | 10.1 | 36.1 | 1.39 |
| acid | | | | |
| Glutamic acid | 30.9 | 9.9 | 40.78 | 1.32 |
| Humic acid | 17.3 | 28.0 | 45.3 | 2.62 |
| Saccharose | 31.6 | 10.5 | 42.1 | 1.33 |

Moreover, various types of waste and industrial by-products were analyzed for elemental carbon with the method according to the invention. As has been expected, the organic fraction prevails in sewage whereas carbon from combustion slags is present in predominantly elemental form. Likewise, soils exhibit a considerable amount of elemental carbon, which seems to be logic insofar as anthropogenically disturbed top soils (which are the main concern of the waste discussion) are often mixed with combustion slags.

The method according to the invention could be easily reproduced all the time, not only with respect to the parameter TOC (i.e. the sum of AOC and RC), but also with respect to the two individual parameters. Thus the pyrolysis process takes place in a controlled and reproducible manner. This is confirmed by the following measurement results:

| | AOC | SDr | RC | SDr | AOC corr. |
|---|---|---|---|---|---|
| Sample | [weight %] | [%] | [weight %] | [%] | [weight %] |
| Sludge | 22.8 | 2.4 | 9.71 | 1.85 | 29.7 |
| Combustion | 1.29 | 3.27 | 9.73 | 0.63 | 1.64 |
| slag | | | | | |
| Coal | 15.7 | 3.42 | 49.1 | 1.32 | 20.6 |
| Soil | 13.2 | 1.80 | 25.1 | 1.08 | 17.2 |

Hence, the above-described method with the corresponding apparatus considerably simplifies the performance of the VGB method. Automatic sample supply and determination of AOC and EC reduce the analyzing time from about 60 minutes to 10 minutes.

What is claimed is:

1. A method for determining degradable organic carbon, comprising the following steps:
feeding the sample to a combustion tube together with a carrier gas;
treating the sample with inert gas in the combustion tube at at least 850° C.;
conducting the obtained pyrolysis gas, which contains the AOC, across a metal oxide as an oxygen donor for oxidizing purposes without adding oxygen;
oxidizing the AOC while the metal oxide is reduced;
measuring the AOC by means of a detector while the CO₂ peak is detected;
switching the carrier gas from inert gas to O₂ once the CO₂ peak has passed;
burning the RC in the O₂ atmosphere while the metal oxide is regenerated; and
measuring the RC by means of a detector.

2. The method according to claim 1, wherein the sample is introduced into a vertically arranged combustion tube.

3. The method according to claim 2, wherein the sample is introduced into the combustion tube by means of a gripper.

4. The method according to claim 3 wherein the oxygen is directly fed to the sample via a lance a the end of which a gripper is arranged.

5. The method according to claim 1, wherein the metal oxide is copper oxide.

6. The method according to claim 1, wherein the metal oxide is tungsten oxide.

7. The method according to claim 1, wherein the metal oxide is cerium dioxide.

8. The method according to claim 1, wherein the AOC is measured by means of an IR detector.

9. The method according to claim 1, wherein the RC is measured by means of an IR detector.
